(19) United States
(12) Patent Application Publication
(10) Pub. No.: US 2015/0045733 A1
(43) Pub. Date: Feb. 12, 2015

(54) Multi-Layered Silicone Pump Segment to Address Bulging

(71) Applicant: CareFusion 303, Inc., San Diego, CA (US)

(72) Inventor: Vincent Bavaro, San Diego, CA (US)

(73) Assignee: CAREFUSION 303, INC., San Diego, CA (US)

(21) Appl. No.: 13/961,838

(22) Filed: Aug. 7, 2013

(51) Int. Cl. A61M 5/142; A61L 29/08; A61L 29/14

(52) U.S. Cl. CPC A61M 5/142; A61L 29/14; A61L 29/085; A61M 2205/0216
USPC 604/151

(57) ABSTRACT

A fluid infusion system that is configured to infuse fluid from a source to a patient. The fluid flows through tubing formed of one or more tube segments that define a fluid lumen. At least a portion of the tubing has a multi-layered configuration including an outer layer and an inner layer. The outer layer has one or more characteristics that are particularly suited for an outer layer such as to resist the environmental conditions that the tubing may experience. The inner layer is formed of a material that is compliant and resilient such that the inner layer may adapt to variations in fluid pressure during pumping of fluid through the tubing

### BACKGROUND

Physicians and other medical personnel apply intravenous (“IV”) infusion therapy to treat various medical complications in patients. IV infusion therapy typically involves infusing medical fluids, such as medications, drugs, or nutrients, from a fluid supply or container, such as a bag or bottle, through the tube of a fluid administration set to a cannula inserted into a patient's blood vessel.

The fluid administrations set includes a section of tubing referred to as a pump segment. A pump mechanism acts on the pump segment portion of the tubing to cause fluid flow through the tube toward the patient. The tubing may be formed of resilient material, such as silicone. The pump segment portion of the tubing may sometimes undergo extreme deformation, such as bulging, as a result of a high fluid pressure situation, such as during a bolus injection from a small volume syringe. It is desirable that the tubing either not have such extreme deformation. Or, should such extreme deformation occur, it is desirable that the tubing returns afterward to its default shape.

### SUMMARY

Disclosed is a fluid infusion system that is configured to infuse fluid from a source to a patient. The fluid flows through tubing formed of one or more tube segments that define a fluid lumen. At least a portion of the tubing has a multi-layered configuration including an outer layer and an inner layer. The outer layer has one or more characteristics that are particularly suited for an outer layer such as to resist the environmental conditions that the tubing may experience. For example, the outer layer may be formed of a material that is particularly tough and cut-resistant. The inner layer is formed of a material that is compliant and resilient such that the inner layer may adapt to variations in fluid pressure during pumping of fluid through the tubing.

In one aspect, there is disclosed a medicant infusion pump system, comprising: a tubing having an outermost layer formed of a cut-resistant material and an innermost layer formed of a material that is more resilient than the outermost layer; and a pump mechanism mechanically coupled to the tubing and configured to act on at least a portion of the tubing to cause fluid flow through the tubing.

In another aspect, there is disclosed a medicant infusion pump system, comprising: a tubing having an outermost layer formed of a cut-resistant material, an intermediate layer formed of a material that is more resilient than the outermost layer, and an innermost layer formed of a material having low migration properties; and a pump mechanism mechanically coupled to the tubing and configured to act on at least a portion of the tubing to cause fluid flow through the tubing.

In another aspect, there is disclosed An infusion tubing configured for use with an infusion pump system, the tubing comprising: a generally cylindrical outermost layer formed of a cut-resistant material; and an innermost layer positioned concentrically within the outermost layer, the innermost layer formed of a material that is more resilient than the outermost layer, wherein the innermost layer defines an internal lumen through which a medicant may flow.

In another aspect, there is disclosed a medicant infusion pump system, comprising: a tubing having an outermost layer formed of a material having shape recovery characteristics, an intermediate layer formed of a material that has lower moisture permeability than another layer of the tubing, and an innermost layer formed of a material having low migration properties; and a pump mechanism mechanically coupled to the tubing and configured to act on at least a portion of the tubing to cause fluid flow through the tubing.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1 is a front view of a patient care system having four fluid infusion pumps, each of which is connected to a respective fluid supply for pumping the contents of the fluid supply to a patient;

FIG. 2 is an enlarged view of a portion of the patient care system of FIG. 1 showing two of the fluid infusion pumps mounted at either side of a programming module, and the displays and control keys of each, with the programming module being capable of programming both infusion pumps;

FIG. 3 is a perspective view of one of the fluid infusion pumps of FIGS. 1 and 2 with its front door in the open;

FIG. 4 shows a cross-sectional view of a pump segment portion of infusion tubing.

FIG. 5 shows a cross-sectional view of another embodiment of the pump segment portion of the tubing.

FIG. 6 shows a cross-sectional view of another embodiment of the pump segment portion of the tubing.

FIG. 7 shows a chemical reaction for a platinum initiated cure in PDMS.

FIG. 8 shows a chemical reaction for a peroxide initiated cross-linking of PDMS.

FIG. 9A shows deformation characteristics of a platinum cured tube with respect to volume (y-axis) vs. pressure inside tubing (x-axis) for both a default state of a tube and after the tube has experienced multiple deformation cycles on the tube.

FIG. 9B shows deformation characteristics of a peroxide cured tube with respect to volume (y-axis) vs. pressure inside tubing (x-axis) for both a default state of a tube and after the tube has experienced multiple deformation cycles on the tube.

### DETAILED DESCRIPTION

Disclose is a fluid infusion system that is configured to infuse fluid from a source to a patient. The fluid flows through tubing formed of one or more tube segments that define a fluid lumen. At least a portion of the tubing has a multi-layered configuration including an outer layer and an inner layer. The outer layer has one or more characteristics that are particularly suited for an outer layer such as to resist the environmental conditions that the tubing may experience. For example, the outer layer may be formed of a material that is particularly tough and cut-resistant. The inner layer is formed of a material that is compliant and resilient such that the inner layer may adapt to variations in fluid pressure during pumping of fluid through the tubing. This is described in more detail below.

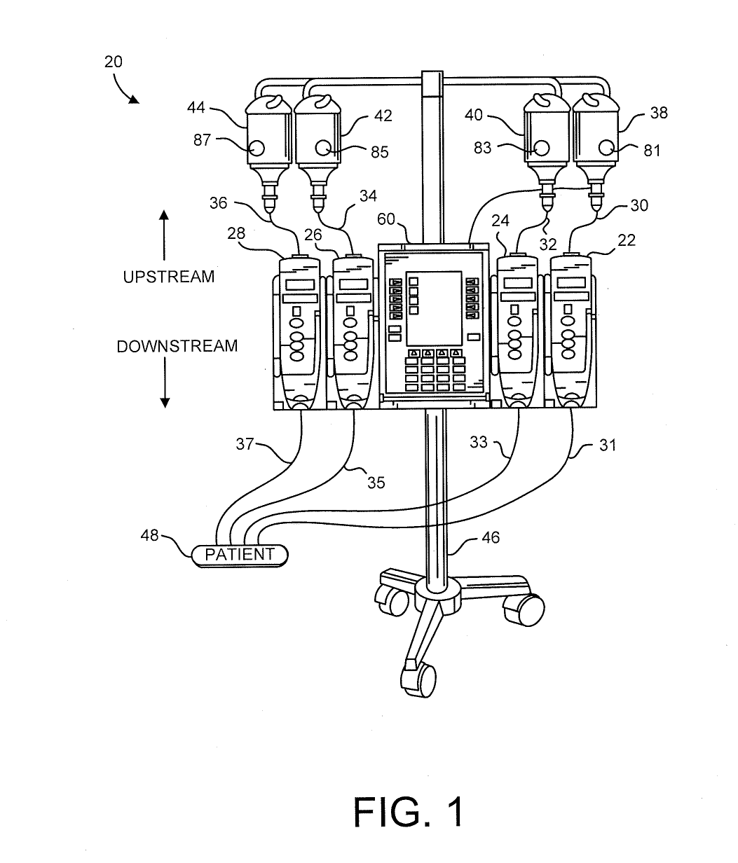

Referring now in more detail to the drawings in which like reference numerals refer to like or corresponding elements among the several views, there is shown in FIG. 1 a patient care system 20 having four infusion pumps 22, 24, 26, and 28 each of which is fluidly connected with an upstream fluid line 30, 32, 34, and 36, respectively. Each of the four infusion pumps 22, 24, 26, and 28 is also fluidly connected with a downstream fluid line 31, 33, 35, and 37, respectively. The fluid lines can be any type of fluid conduit, such as tubing, through which fluid can flow. At least a portion of one or more of the fluid lines may be constructed with a multi-layered configuration as described herein.

Fluid supplies 38, 40, 42, and 44, which may take various forms but in this case are shown as bottles, are inverted and suspended above the pumps. Fluid supplies may also take the form of bags or other types of containers. Both the patient care system 20 and the fluid supplies 38, 40, 42, and 44 are mounted to a roller stand or IV pole 46.

A separate infusion pump 22, 24, 26, and 28 is used to infuse each of the fluids of the fluid supplies into the patient. The infusion pumps are flow control devices that will act on the respective fluid line to move the fluid from the fluid supply through the fluid line to the patient 48. Because individual pumps are used, each can be individually set to the pumping or operating parameters required for infusing the particular medical fluid from the respective fluid supply into the patient at the particular rate prescribed for that fluid by the physician. Such medical fluids may comprise drugs or nutrients or other fluids.

Typically, medical fluid administration sets have more parts than are shown in FIG. 1. Many have check valves, drip chambers, valved ports, connectors, and other devices well known to those skilled in the art. These other devices have not been included in the drawings so as to preserve clarity of illustration. In addition, it should be noted that the drawing of FIG. 1 is not to scale and that distances have been compressed for the purpose of clarity. In an actual setting, the distance between the bottles 38, 40, 42, and 44 and the infusion pump modules 22, 24, 26, and 28 could be much greater.

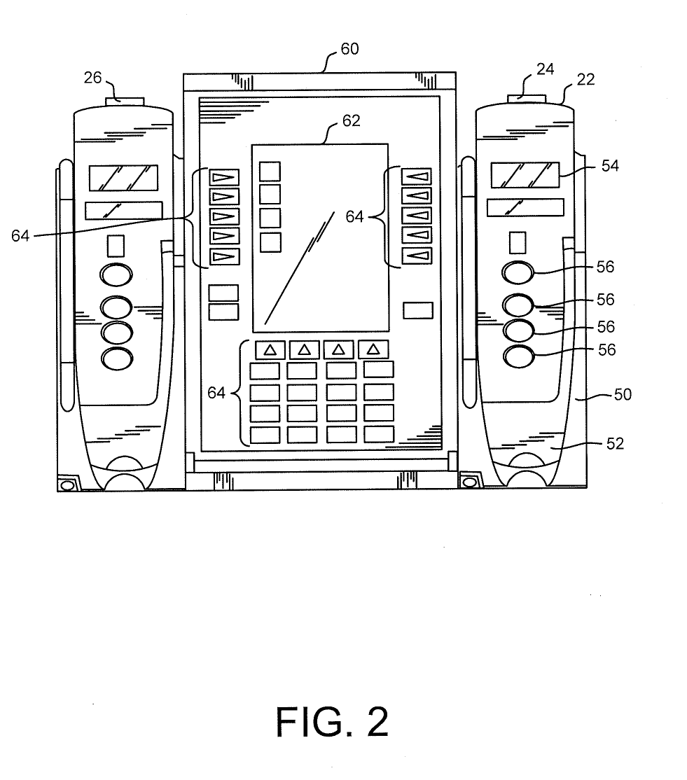

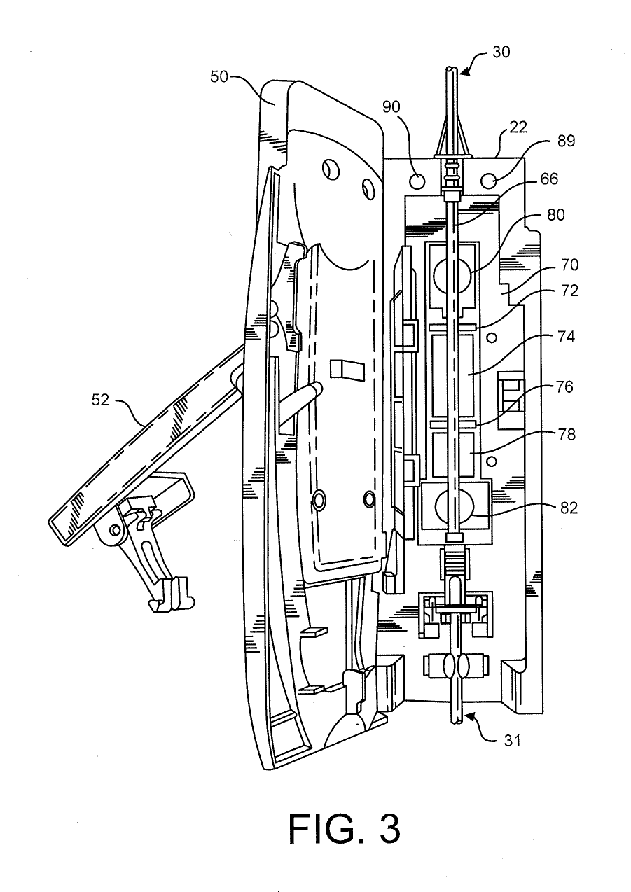

Referring now to FIG. 2, an enlarged view of the front of the infusion pump 24 is shown. The pump includes a front door 50 and a handle 52 that operates to lock the door in a closed position for operation and to unlock and open the door for access to the internal pumping and sensing mechanisms and to load administration sets for the pump. When the door is open, the tube can be connected with the pump, as will be shown in FIG. 3. When the door is closed, the tube is brought into operating engagement with the pumping mechanism, the upstream and downstream pressure sensors, and the other equipment of the pump. A display 54, such as an LED display, is located in plain view on the door in this embodiment and may be used to visually communicate various information relevant to the pump, such as alert indications (e.g., alarm messages). Control keys 56 exist for programming and controlling operations of the infusion pump as desired. The infusion pump 24 also includes audio alarm equipment in the form of a speaker (not shown).

In the embodiment shown, a programming module 60 is attached to the left side of the infusion pump 24. Other devices or modules, including another infusion pump, may be attached to the right side of the infusion pump 24, as shown in FIG. 1. In such a system, each attached pump represents a pump channel of the overall patient care system 20. In one embodiment, the programming module is used to provide an interface between the infusion pump 24 and external devices as well as to provide most of the operator interface for the infusion pump 24.

The programming module 60 includes a display 62 for visually communicating various information, such as the operating parameters of the pump 24 and alert indications and alarm messages. The programming module 60 may also include a speaker to provide audible alarms. The programming module or any other module also has various input devices in this embodiment, including control keys 64 and a bar code or other scanner or reader for scanning information from an electronic data tag relating to the infusion, the patient, the care giver, or other. The programming module also has a communications system (not shown) with which it may communicate with external equipment such as a medical facility server or other computer and with a portable processor, such as a handheld portable digital assistant (“PDA), or a laptop-type of computer, or other information device that a care giver may have to transfer information as well as to download drug libraries to a programming module or pump.

The communications system may take the form of a radio frequency (“RF”) (radio frequency) system, an optical system such as infrared, a Blue Tooth system, or other wired or wireless system. The bar code scanner and communications system may alternatively be included integrally with the infusion pump 24, such as in cases where a programming module is not used, or in addition to one with the programming module. Further, information input devices need not be hard-wired to medical instruments, information may be transferred through a wireless connection as well.

FIG. 2 includes a second pump module 26 connected to the programming module 60. As shown in FIG. 1, more pump modules may be connected. Additionally, other types of modules may be connected to the pump modules or to the programming module.

Turning now to FIG. 3, an infusion pump 22 is shown in perspective view with the front door 50 open, showing the upstream fluid line 30 and downstream fluid line 31 in operative engagement with the pump 22. The infusion pump 22 directly acts on a tube 66 (also referred to as a pump segment) that connects the upstream fluid line 30 to the downstream fluid line 31 to form a continuous fluid conduit, extending from the respective fluid supply 38 (FIG. 1) to the patient 48, through which fluid is acted upon by the pump to move fluid downstream to the patient. Specifically, a pumping mechanism 70 acts as the flow control device of the pump to move fluid though the conduit. The upstream and downstream fluid lines and/or tube 66 may be coupled to a pump cassette or cartridge that is configured to be coupled to the pump 2, such as the type described in co-pending U.S. patent application Ser. No. 13/827,775, which is incorporated by reference herein.

The type of pumping mechanism may vary and may be for example, a multiple finger pumping mechanism. For example, the pumping mechanism may be of the “four finger” type and includes an upstream occluding finger 72, a primary pumping finger 74, a downstream occluding finger 76, and a secondary pumping finger 78. The “four finger” pumping mechanism and mechanisms used in other linear peristaltic pumps operate by sequentially pressing on a segment of the fluid conduit by means of the cam-following pumping fingers and valve fingers 72, 74, 76, and 78. The pressure is applied in sequential locations of the conduit, beginning at the upstream end of the pumping mechanism and working toward the downstream end. At least one finger is always pressing hard enough to occlude the conduit. As a practical matter, one finger does not retract from occluding the tubing until the next one in sequence has already occluded the tubing; thus at no time is there a direct fluid path from the fluid supply to the patient. The operation of peristaltic pumps including four finger pumps is well known to those skilled in the art and no further operational details are provided here.

In this particular embodiment, FIG. 3 further shows a downstream pressure sensor 82 included in the pump 22 at a downstream location with respect to the pumping mechanism. The downstream pressure sensor 82 is mounted to the flow control device 70 and is located adjacent and downstream in relation to the flow control device. The downstream pressure sensor is located downstream from the flow control device, that is, at a location between the patient 48 (FIG. 1) and the flow control device, so that the connection of the correct fluid supply with the correct pump may be verified before any fluid is pumped to the patient.

With reference still to FIG. 3, an upstream pressure sensor 80 may also be included in the pump 22. The upstream pressure sensor is assigned to the flow control device or pumping mechanism 70 and, in this embodiment, is further provided as an integral part of the pump 22. It is mounted to the flow control device 70 and is located adjacent and upstream in relation to the flow control device. The upstream pressure sensor is located upstream from the flow control device, that is, at a location between the fluid supply 38 (FIG. 1) and the flow control device, so that the connection of the correct fluid supply with the correct pump may be verified before any fluid is pumped to the patient.

### Multi-Layered Pump Segment

As discussed, at least a portion of the tubing through which fluid flow to the patient is configured with one or more properties that are suited to a particular use of the tubing. In an embodiment, an outermost portion of the tubing is formed of a material that is tough and resistant to cutting while an innermost portion of the tubing (the portion that defines an internal lumen) is manufactured of a material that has resilient properties.

In an embodiment, the pump segment portion of the tubing (such as the tube 66 in FIG. 3) is a multi-layered tube formed of two or more layers of material. For example, an outermost layer is resistant to cutting or deformation that would be harmful to the tubing. This permits the outermost layer to be resistant to environmental conditions that the tubing may encounter. An inner or innermost layer is more elastic than the outermost layer (or other layers) of the tubing and is configured to maintain desirable compliance and cyclic hysteresis properties.

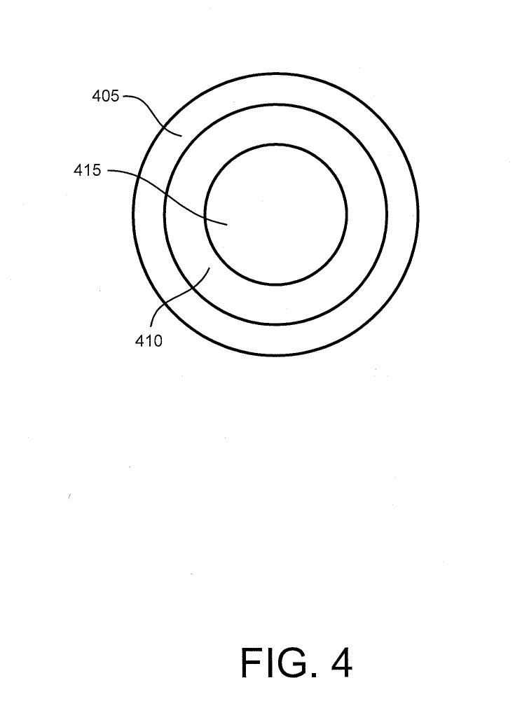

FIG. 4 shows a cross-sectional view of the pump segment portion of the tubing. An outermost layer 405 is formed of a cut resistant material, such as platinum cured material. This outermost layer 405 is tough and cut-resistant. An innermost layer 410 defines an internal lumen 415 for fluid flow is formed of a material that is more elastic than the outermost layer 405. The innermost layer 415 desirably has different hysteresis characteristics relative to the outermost layer 405. That is the innermost layer 415 has an increased ability to return to a default shape after undergoing deformation. In an embodiment, the innermost layer 410 and the outermost layer 405 are the only layers of the tubing such that the tubing has only two layers. In another embodiment, the tubing has more than two layers.

At least one difference between the layers of the tubing is the cross-link density of the layers. The tubing may be manufactured, for example, by co-extrusion process such as by using a pair of extruders that feed the different materials of the layers into the formed tubing.

The material that is used to form the layers of the tubing may vary. In an embodiment, the outermost layer 405 is formed of a platinum cured resin. In an embodiment, the outermost layer is a platinum cured silicone (polydimethylsiloxane or PDMS) such as a BIOSIL 6 platinum cured material. For the tubing, source polymers are typically long chains of polydimethylsiloxane, either methyl or hydrogen-substituted, and vinyl-functional polydimethylsiloxane gums.

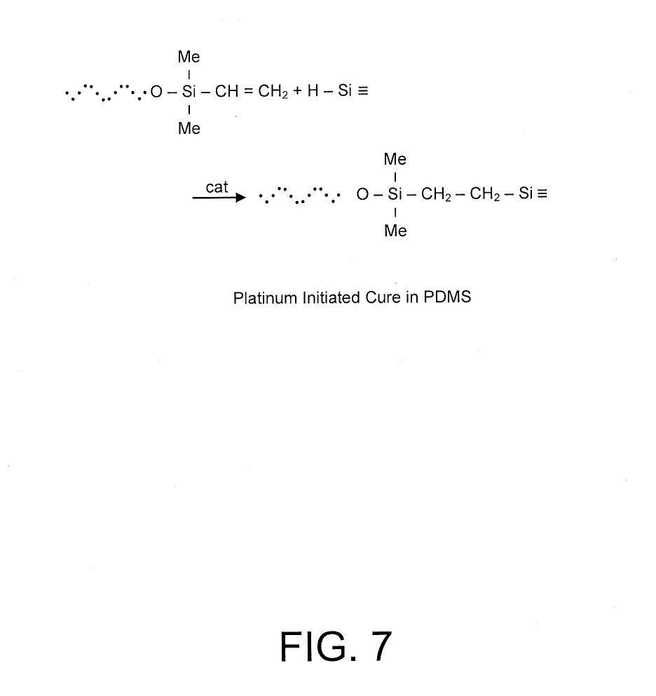

During manufacture, a cure system employs vinyl-functional polymers, fluid with Si—H groups, and a metal complex catalyst, such as platinum. The reaction is shown in FIG. 7. In the reaction shown in FIG. 7, the (H—Si═) Hydride fluid has multiple hydrogen groups on the chain and crosslink to the gums is only through this fluid. Pursuant to the platinum cured process, the number of crosslinks is determined by the number of vinyl groups on the gum and the number of hydride groups on the fluid. The crosslinks are slightly further apart but predictable and consistent.

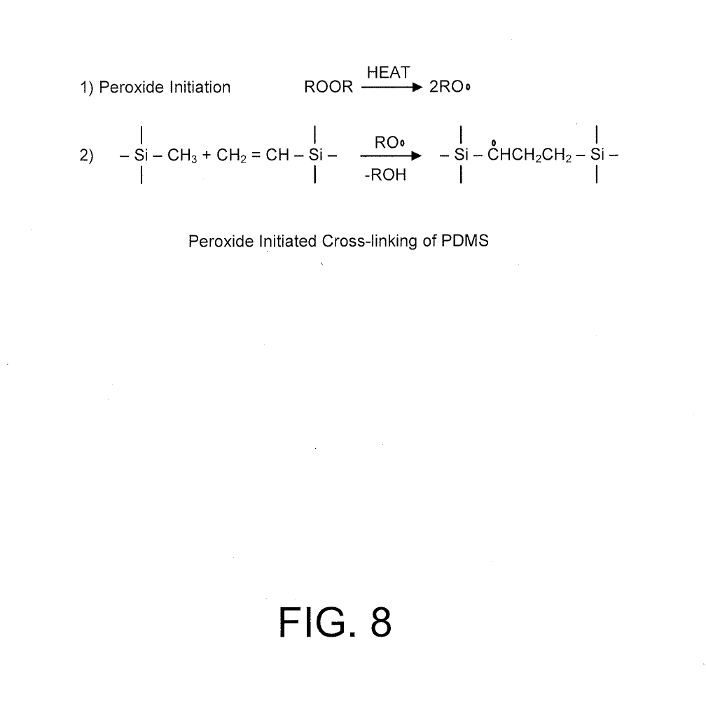

In an embodiment, the innermost layer is formed of a peroxide cured resin PDMS, such as SHINITSU peroxide cured material. FIG. 8 shows the reaction for the peroxide cured material. In the peroxide cured material, the reaction requires only a vinyl and a methyl group to achieve a crosslink. Thus, the distance between the gums are very short (a three carbon chain.) They are also less predictable and the numbers of crosslinks are determined by the amount of peroxide used. The following reference is incorporated by reference in its entirety: “Fascinating Silicone Chemistry Corner-Peroxide and Platinum Addition Cure”, Dow Corning Corp, 2013, obtainable at http://www.dowcorning.com/content/discover/discoverchem/si-finishing.aspx?e=Silicone+Manufacturing.

Although the physical properties of the platinum cured material and the peroxide cured material are similar, the cross linking as formed in the reaction of FIG. 7 causes the material to expand more due to the greater chain length between the crosslinks, which causes larger round shaped bubbles in the tubing (i.e., more elongated at the same pressure or more compliant). The reaction of FIG. 8 results in less expansion due to the shorter chain distance between crosslinks. By way of practical example, bubble formation starts at around 25 psi in tubing made solely of either material, but the platinum cured material expands more before breaking.

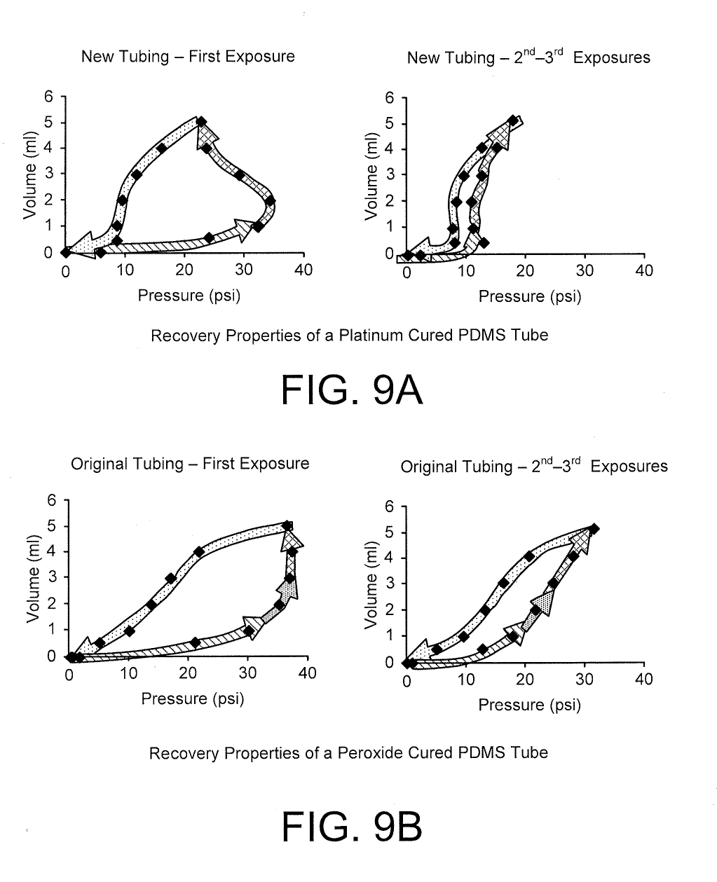

FIG. 9A shows the deformation characteristics of the platinum cured tube (with respect to volume (y-axis) vs. pressure inside tubing (x-axis)) that illustrate the recovery or hysteresis properties while FIG. 9B shows the same for the peroxide cured material. The graphs to the right of FIGS. 9A and 9B show the effect of multiple deformation cycles on said tube, which is an indication of the tubes' resilience to cyclic fatigue. The difference between FIGS. 9A left graph and 9A right graph indicates that the material yields at lower pressure, 30 psi vs 10 psi respectively, upon subsequent exposure to force. This mode is undesirable in clinical practice as the tube is more compliant to a subsequent bolus or flush injection with low volume syringes. Whereas, the difference between FIGS. 9B left graph and 9B right graph is less pronounced upon subsequent exposure to force.

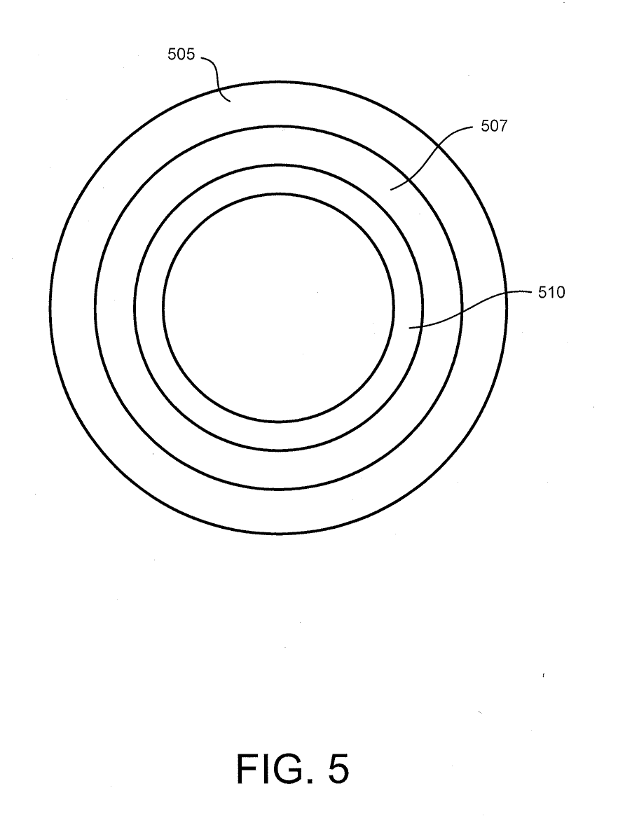

FIG. 5 shows a cross-sectional view of another embodiment of the pump segment portion of the tubing. An outermost layer 505 is formed of a cut resistant material, such as platinum catalyzed material. This outermost layer 505 is tough and cut-resistant. An intermediate layer 507 is interposed between the outermost layer 505 and an innermost layer 510 that defines an internal lumen 515 for fluid flow. The intermediate layer 507 is formed of a material that is more elastic than the outermost layer 505. The intermediate layer 507 desirably has different hysteresis characteristics relative to the one or more of the other layers. That is the intermediate layer 507 has an increased ability to return to a default shape after undergoing deformation. In an embodiment, the intermediate layer 507 is formed of a peroxide catalyzed material. The innermost layer 510 is configured for low migration characteristics and may be formed of, for example, a platinum cured material.

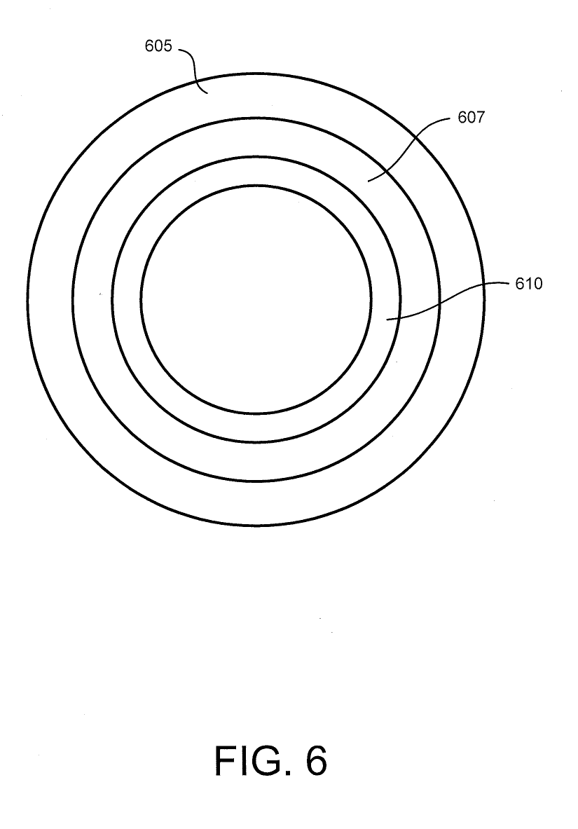

FIG. 6 shows a cross-sectional view of another embodiment of the pump segment portion of the tubing. An outermost layer 605 is formed of a material that is particularly suited for shape recover after deformation. The outermost layer 605 may be formed of a peroxide catalyzed material. An intermediate layer 607 is interposed between the outermost layer 605 and an innermost layer 610 that defines an internal lumen 515 for fluid flow. The intermediate layer 507 is formed of a material that has low moisture permeability relative to the other layers. The innermost layer 510 is configured for low migration characteristics and may be formed of, for example, a platinum cured material.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flow(s) when depicted in the accompanying figures and/or described herein do not necessarily require the particular order shown, or sequential order, to achieve desirable results. Other implementations may be within the scope of the following claims.

1. A medicant infusion pump system, comprising:
a tubing having an outermost layer formed of a cut-resistant material and an innermost layer formed of a material that is more resilient than the outermost layer;
a pump mechanism mechanically coupled to the tubing and configured to act on at least a portion of the tubing to cause fluid flow through the tubing.

2. A system as in claim 1, wherein the outermost layer comprises a platinum cured resin.

3. A system as in claim 1, wherein the innermost layer comprises a peroxide cured resin.

4. A system as in claim 1, wherein the innermost and outermost layers are the only layers of the tubing.

5. A system as in claim 1, wherein the innermost and outermost layers have different hysteresis properties.

6. A system as in claim 1, wherein the tubing is manufactured pursuant to a co-extrusion process.

7. A system as in claim 1, wherein the pump mechanism acts directly onto the tubing.

8. A system as in claim 1, wherein the outermost layer comprises Biosil platinum cured material.

9. A system as in claim 1, wherein the innermost layer comprises a Shinitsu peroxide cured material.

10. A system as in claim 1, wherein the tubing comprises more than two layers.

11. A medicant infusion pump system, comprising:
a tubing having an outermost layer formed of a cut-resistant material, an intermediate layer formed of a material that is more resilient than the outermost layer, and an innermost layer formed of a material having low migration properties;
a pump mechanism mechanically coupled to the tubing and configured to act on at least a portion of the tubing to cause fluid flow through the tubing.

12. A system as in claim 11, wherein the outermost layer comprises a platinum catalyzed material.

13. A system as in claim 11, wherein the innermost layer comprises a platinum cured material.

14. A system as in claim 11, wherein the intermediate layer comprises a peroxide catalyzed material.

15. An infusion tubing configured for use with an infusion pump system, the tubing comprising:
a generally cylindrical outermost layer formed of a cut-resistant material; and
an innermost layer positioned concentrically within the outermost layer, the innermost layer formed of a material that is more resilient than the outermost layer, wherein the innermost layer defines an internal lumen through which a medicant may flow.

16. A tubing as in claim 15, wherein the outermost layer comprises a platinum cured resin.

17. A tubing as in claim 15, wherein the innermost layer comprises a peroxide cured resin.

18. A tubing as in claim 15, wherein the innermost and outermost layers are the only layers of the tubing.

19. A tubing as in claim 15, wherein the innermost and outermost layers have different hysteresis properties.

20. A tubing as in claim 15, wherein the tubing is manufactured pursuant to a co-extrusion process.

21. A tubing as in claim 15, wherein the outermost layer comprises Biosil platinum cured material.

22. A tubing as in claim 15, wherein the innermost layer comprises a Shinitsu peroxide cured material.

23. A tubing as in claim 1, wherein the tubing comprises more than two layers.

24. A medicant infusion pump system, comprising:
a tubing having an outermost layer formed of a material having shape recovery characteristics, an intermediate layer formed of a material that has lower moisture permeability than another layer of the tubing, and an innermost layer formed of a material having low migration properties;
a pump mechanism mechanically coupled to the tubing and configured to act on at least a portion of the tubing to cause fluid flow through the tubing.

25. A system as in claim 23, wherein the outermost layer comprises a peroxide catalyzed material.

26. A system as in claim 25, wherein the innermost layer comprises a platinum cured material.
